(19) United States
(12) Patent Application Publication
(10) Pub. No.: US 2004/0038318 A1
(43) Pub. Date: Feb. 26, 2004

(54) Creatine kinase isoenzyme determination in multiplexed assays

(76) Inventor: Michael L. Bell, Fullerton, CA (US)

Correspondence Address: PATENT LEGAL DEPARTMENT/A-42-C, BECKMAN COULTER, INC., 4300 N. HARBOR BOULEVARD, BOX 3100, FULLERTON, CA, 92834-3100, US

(21) Appl. No.: 10/226,876

(22) Filed: Aug. 23, 2002

(51) Int. Cl. G01N 33/573

(52) U.S. Cl. 435/7.4

(57) ABSTRACT

This invention relates to a reagent set, as well as to a method, for carrying out simultaneous analyses of multiple isoenzymes in a test sample, particularly a bodily fluid. The invention is especially useful for measuring creatine kinase isoenzymes in particle, or bead, based multiplexed assay systems.

### BACKGROUND

Creatine kinase, also known as creatine phosphokinase, is an enzyme found predominantly in the heart, brain and skeletal muscle. Creatine kinase is an isoenzyme, found in three dimeric forms: a muscle enzyme (MM) consisting of two identical M subunits, a brain enzyme (BB) consisting of two identical B subunits, and the third, hybrid enzyme (MB) consisting of one M subunit and one B subunit.

The dimeric creatine kinase isoenzymes are involved in maintaining intracellular ATP levels, particularly in high-energy-demand tissues. The creatine kinase BB isoenzyme, known as creatine kinase 1 or creatine kinase BB, is found in smooth muscle, brain and nerve. The creatine kinase hybrid MB isoenzyme, known as creatine kinase 2 or creatine kinase MB, is found in the heart. Finally, the creatine kinase MM isoenzyme, known as creatine kinase 3 or creatine kinase MM, is found in striated muscle.

The creatine kinase isoenzymes are important markers for various diseases and pathological conditions, and damage to organs such as the brain, lungs, heart or muscle may cause the corresponding isoenzyme to leak into the bloodstream. The normal total creatine kinase level is approximately 35 to 190 units per liter, with approximately 96% to 100% of the total creatine kinase being creatine kinase 3, the remainder being creatine kinase 2. Elevated creatine kinase levels may be indicative of disease or organ damage.

For example, abnormally high levels of creatine kinase 1 may indicate brain cancer, head trauma and/or bleeding around the brain, lung damage, damage from prolonged seizures or stroke. Abnormally high levels of creatine kinase 2 may indicate heart attack or other trauma to the heart, or electrical injuries. Finally, abnormally high levels of creatine kinase 3 may indicate heart attack, muscle damage, intramuscular injuries, muscular dystrophy, myositis, convulsions, and rhabdomyolysis.

Creatine kinase 2 is a particularly important marker, as levels of the isoenzyme rise 3 to 6 hours after a myocardial infarction, or heart attack. If no further myocardial damage occurs, the level of creatine kinase 2 peaks at 12 to 24 hours, and returns to normal 12 to 48 hours after infarction. Creatine kinase levels generally do not rise with chest pain caused by angina, pulmonary embolism or congestive heart failure, and thus are useful in evaluating chest pain and in diagnosing myocardial infarction.

Creatine kinase tests are also useful for detecting early dermatomyositis, to determine the extent of muscular disease, to distinguish malignant hyperthermia from a postoperative infection, and to help discover carriers of muscular dystrophy.

Generally, a total creatine kinase test is about 70% accurate, while isoenzyme testing for the individual types of creatine kinase is about 90% accurate.

In assessing a patient's physiological state, a variety of diagnostic techniques in addition to creatine kinase testing have been developed to analyze substances found in the body chemistry that may be indicators of disease or pathological conditions. Typically, it is necessary to carry out each test individually, at a substantial cost and effort.

Recently, however, multiplexed assays have been described in which several analytes may be tested simultaneously, greatly increasing the efficiency, and reducing the cost, of running panels of diagnostic tests. See, for example, van den Engh et al., U.S. Pat. No. 5,747,349 and Chandler et al., U.S. Pat. No. 5,981,180.

Given the importance of creatine kinase tests in assessing a patient's physiological state, it would be useful to include tests for the various creatine kinase isoenzymes in such multiplexed assays.

One way to measure creatine kinase is by providing an assay in which creatine kinase activity (that is, cleaving phosphate groups from creatine phosphate) results in a detectible signal, such as a change in fluorescence. However, creatine kinase activity measurement is problematic in multiplexed systems, because it interacts with other analytes. Creatine kinase requires magnesium ions for activity, and is inhibited by calcium and chloride ions. In multiplexed assays used to evaluate a patient's physiological state, however, magnesium, calcium and chloride ions are among the analytes commonly being measured. Accordingly, it is not practical to provide multiplexed assay conditions in which the magnesium, calcium and chloride concentrations are adjusted to measure creatine kinase activity.

Another way to measure the amount of creatine kinase present in a biological sample is to use an immunoassay in which antibodies to creatine kinase are used to detect and measure the enzyme. However, standard immunological techniques with a single reporter species, as used in multiplexed assays, while useful to detect a single isoenzyme, cannot simultaneously determine total creatine kinase, nor can they separately measure the creatine kinase isoenzymes.

What is needed, therefore, is a method for measuring both total creatine kinase and individual creatine kinase isoenzymes in a single assay, which can be incorporated into a multiplexed assay system.

### SUMMARY OF THE INVENTION

It is an advantage of the present invention to provide an assay, including assay reagents and assay method, which enables the contemporaneous measurement of multiple isoenzymes in a single test procedure.

It is a further advantage of the present invention to provide an assay for multiple isoenzymes, which can be incorporated into a particle, or bead, based multiplexed assay system to permit contemporaneous measurement of analytes in addition to the multiple isoenzymes. The present invention is particularly useful for measuring creatine kinase isoenzymes in multiplexed assays.

The present invention provides a reagent set for measuring creatine kinase (CK) isoenzymes CK-1, CK-2, and CK-3 in a test sample, the reagent set comprising:

creatine kinase B (CKB) capture particles comprising a plurality of sample-insoluble particles having associated therewith immobilized antibody adapted to interact with a B subunit of creatine kinase;

creatine kinase M (CKM) capture particles comprising a plurality of sample-insoluble particles having associated therewith immobilized antibody adapted to interact with an M subunit of creatine kinase, wherein the CKM capture particles are optically distinguishable from the CKB capture particles;

a plurality of CKB reporters, adapted to interact with a B subunit of creatine kinase, to thereby produce a first fluorescent signal; and

a plurality of CKM reporters, adapted to interact with M subunit of creatine kinase, to thereby produce a second fluorescent signal.

The present invention further provides a method for measuring creatine kinase isoenzymes CK-1, CK-2, CK-3 as well as total creatine kinase content in a test sample, the method comprising the steps of:

(a) mixing the test sample with a reagent set comprising:

(1) CKB capture particles comprising a plurality of sample-insoluble particles having associated therewith immobilized antibody adapted to interact with a B subunit of creatine kinase and further comprising coding indicia which confer uniquely identifying optical properties on the CKB capture particles;

(2) CKM capture particles comprising a plurality of sample-insoluble particles having associated therewith immobilized antibody adapted to interact with an M subunit of creatine kinase and further comprising coding indicia which confer uniquely identifying optical properties on the CKM capture particles;

(3) a plurality of CKB reporters, each adapted to interact with a CKB subunit, to thereby produce a first fluorescent signal; and

(4) a plurality of CKM reporters, each adapted to interact with a CKM subunit, to thereby produce a second fluorescent signal;

(b) incubating the resulting mixture for a period of time sufficient for:

(1) the CKB capture particles to interact with B subunit of CK-1 to form a CKB/CK-1 capture particle complex and/or with B subunit of CK-2 to form a CKB/CK-2 capture particle complex;

(2) the CKM capture particles to interact with M subunit of CK-2 to form a CKM/CK-2 capture particle complex and/or with M subunit of CK-3 to form a CKM/CK-3 capture particle complex;

(3) the CKB reporters to interact with B subunit of CK-1 on CKB/CK-1 capture particle complexes and/or with B subunit of CK-2 on CKM/CK-2 capture particle complexes; and

(4) the CKM reporters to interact with M subunit of CK-2 on CKB/CK-2 capture particle complexes and/or with M subunit of CK-3 on CKM/CK-3 capture particle complexes;

(c) reading both the coding optical properties and the fluorescent signal or signals of each capture particle complex individually;

(d) storing the measured fluorescent signal of both the CKB capture particle complexes and the CKM capture particle complexes according to the coding optical properties read from the complexes; and

(e) processing the stored measurements for the CKB capture particle complexes to obtain an assay result for CK-1 and for CK-2 and processing the stored measurements for the CKM capture particle complexes to obtain an assay result for CK-2 and for CK-3;

whereby a complete chemical analysis of the creatine kinase content of the test sample is obtained.

Other advantages and novel features of the present invention will become apparent from the following detailed description of the invention and the accompanying drawings.

### BRIEF DESCRIPTION OF THE DRAWINGS

FIG. 1 is a schematic representation of a mixture of the reagents of the present invention with a test sample containing creatine kinase isoenzymes CK-1, CK-2, and CK-3.

FIG. 2 is a schematic representation of a CKB capture particle complex containing CK-1 and a CKM capture particle complex containing CK-2 and CK-3, with attached reporter antibodies.

FIG. 3 is a schematic representation of a reading device of the present invention, showing a CKB capture particle complex passing through a beam from a coding laser.

FIG. 4 is a schematic representation of a reading device of the present invention, showing the CKB capture particle complex passing through a beam from an assay laser.

### DETAILED DESCRIPTION

Brief Overview of the Methods of the Present Invention.

The present invention extends the utility of multiplexed assays by providing an immunoassay that can distinguish between or among various isoenzymes to evaluate the concentration of each isoenzyme. In particular, the present invention provides a method of determining the concentration of the various creatine kinase isoenzymes.

In accordance with the present invention, particles coupled to antibodies (hereinafter, capture particles) directed to the creatine kinase subunits are mixed with biological samples, binding creatine kinase present in the sample to the capture particles. One type of capture particle (hereinafter, CKB capture particles) is coupled to antibodies directed specifically against the creatine kinase B subunit, and, accordingly, binds both creatine kinase 1 (CK-1), containing two B subunits, and creatine kinase 2 (CK-2), containing a B subunit and an M subunit. A second type of capture particle (hereinafter, CKM capture particles) is coupled to antibodies directed specifically against the creatine kinase M subunit and, accordingly, binds both CK-2, containing a B subunit and an M subunit, and creatine kinase 3 (CK-3), containing two M subunits. Each of the two types of capture particles includes coding indicia, to permit identification of the capture particle as either a CKB capture particle or a CKM capture particle.

Two types of reporter antibodies, each distinctly labeled, are also used in the method of the present invention. The first reporter antibody (hereinafter, CKB reporter antibody) has specificity for the creatine kinase B subunit and is labeled, for example, with a first fluor. The second reporter antibody (hereinafter, CKM reporter antibody) has specificity for the creatine kinase M subunit and is labeled, for example, with a second fluor.

When mixed with the capture particles and the biological sample, the reporter antibodies bind to free subunits of creatine kinase; that is, reporter antibody binds to subunits of creatine kinase not already bound to antibody on the capture particles. CKB reporter antibody will bind to the free B subunit of CK-1 on CKB capture particles and to the free B subunit of CK-2 on CKM capture particles. Similarly, CKM reporter antibody will bind to the free M subunit of CK-2 on CKB capture particles and to the free M subunit of CK-3 on CKM capture particles.

The capture particles, with associated creatine kinase and reporter antibodies, are then passed singly through a flow cytometer, or are otherwise similarly detected, where the unique coding indicia identifies the type of capture particle, while the signal channels record the intensity from each of the two fluors of the reporter antibodies. Combining measurements from both types of particles allows computation of the concentration of each of the isoenzyme species. These concentrations can be summed to determine the total creatine kinase concentration.

Thus, for example, if a CKB capture particle is detected in the flow cytometer, the intensity of the first fluor associated with that capture particle provides a measure of CK-1, while the intensity of the second fluor associated with that capture particle provides a measure of CK-2. Likewise, if an CKM capture particle is detected in the flow cytometer, the intensity of the first fluor associated with that capture particle provides a measure of CK-2, while the intensity of the second fluor associated with that capture particle provides a measure of CK-3. The four possible outcomes are summarized in Table 1.

TABLE 1

| | CKB Capture | CKM Capture |
|---|---|---|
| | Particle | Particle |
| CKB Reporter | CK-1 | CK-2 |
| Antibody | | |
| CKM Reporter | CK-2 | CK-3 |
| Antibody | | |

As will be appreciated, the present method permits creatine kinase isoenzyme measurement in multiplexing systems. Advantageously, the present method avoids interference by other analytes in multiplex assays, uses only two particle types and two antibodies, and permits direct determination of each creatine kinase isoenzyme. Additionally, the present invention employs all of the capture particles in the measurement of the clinically critical CK-2, thereby improving confidence in the quality of results.

Analytes. Although the method of the present invention is described with reference to creatine kinase isoenzymes, one of skill in the art will appreciate that the present method is useful with a wide variety of analytes. For example, the present method has general applicability to any isoenzyme families for which antibodies are available that can differentiate between the isoenzymes. Examples of other analytes that can be measured using the present method include, for example, lactate dehydrogenase.

Also, the present method can be used to measure analytes, including creatine kinase, in a variety of biological samples, including but not limited to whole blood, serum, plasma, saliva, urine, lymph, spinal fluid, tears, mucous, semen and other body fluids. Generally, creatine kinase activity is measured in whole blood or blood serum.

Capture Particle. The particles of the instant method are preferably spherical in shape, although other shapes can be used. It is important, however, that all the particles exhibit similar optical characteristics.

Typically, particles useful in the present method are from about 500 nm to about 50 μm in diameter, preferably from about 3 μm to about 15 μm in diameter, and more preferably about 5 μm in diameter. Particles of this size range, with a density typically from about 0.5 to 2.0 grams per milliliter, act as simple liquids when suspended in an aqueous medium, and can be conveyed by conventional fluid transfer techniques, such as pipettes, pumps, and the like.

The particles can be formed from a number of suitable materials, including but not limited to polystyrene, carboxylate modified polystyrene, acrylate, methylacrylate/decamethylacrylate copolymer, polyvinyl chloride, polystyrene/divinylbenzene copolymer and controlled pore glass beads. Acrylate or methacrylate derived particles can be produced by suspension or dispersion polymerization techniques. Other possible bead or particle sources include hydrogel polymer particles, polymerized micelle particles, particles produced by grinding cast films, particles produced by photopolymerization of aqueous emulsions, and particles produced by solvent casting as described in U.S. Pat. Nos. 4,302,166 and 4,162,282.

Particles suitable as a starting material in accordance with practice of the present invention are commercially available from suppliers such as Molecular Probes, Inc. (Eugene, Oreg.), Bangs Laboratories (Indianapolis, Ind.) or Spherotech, Inc. (Libertyville, Ill.).

Particles are coded to include coding indicia, thereby enabling unambiguous identification of the particle type and consequently enabling the detection and analysis system to correlate measurement signals from the particle to the specific analyte with which the particular type of particle interacts. Particles can be coded by varying detectable particle properties. One relatively simple technique for coding particles is to use particles of different sizes. As different size particles move singly through the laser beam in a flow cytometer, each particle size will produce a different, and distinctive, amount of light scattering, allowing identification of the particle type. As is known in flow cytometry, the forward scatter and side scatter signals can be used singly or together to determine particle size.

Alternatively, fluorescent dye can be used to code the capture particles. In one embodiment, one type of capture particle is labeled with fluorescent dye, while the second type of capture particle is left unlabeled. In this embodiment, the presence or absence of fluorescent dye on a capture particle as it passes through the laser beam in a flow cytometer identifies the type of capture particle.

In still other embodiments, the intensity of fluorescence from fluorescent dyes associated with the capture particles, ratios of intensities of fluorescence from multiple fluorescent dyes associated with the capture particles, and fluorescence wavelengths of one or more fluorescent dyes associated with the capture particles can be varied as coding indicia.

Other detectable signals that can serve as coding indicia include number of particles, the addition of magnetic materials, fluorophores that can be quenched so that it gives off no light, and combinations of all of the above. Additional information about useful coding schemes may be found in Fulwyler, U.S. Pat. No. 4,499,052, Coulter Electronics, U.K. Patent No. 1,561,042, Lehnen, U.S. Pat. No. 5,567,627, Saunders, U.S. Pat. No. 3,925,018, and Tripatzis, European Patent No. 126450.

Antibodies. As described above, and in more detail below, the capture particles used in the method of the present invention are associated with antibodies directed to either the creatine kinase B subunit or the creatine kinase M subunit. The antibodies are immobilized on the surface of the particles, preferably by covalent interaction as, for example, with the carboxyl groups of carboxylate modified polystyrene.

Preferably, the same two antibodies, each labeled with a different detectable, and distinguishable, marker such as a fluorescent dye, are also used as reporter antibodies. In use, the antibody on the capture particle binds to one subunit of creatine kinase, while the labeled reporter antibody binds to the second subunit of creatine kinase, forming a “sandwich.”

Typically, the antibody molecules used will be monoclonal antibodies with high affinity constants, preferably with affinity constants of 10⁻⁹ M⁻¹ or more. Polyclonal antibodies, particularly affinity-purified polyclonal antibodies, and antibody fragments can also be used. Importantly, the antibodies used should be specific to one of the creatine kinase subunits, with little or no cross-reactivity to the other subunit. Antibodies specific to the creatine kinase B subunit or to the creatine kinase M subunit are commercially available from Spectral Diagnostics, Inc. (Whitestone, Va.). Methods to prepare antibodies specific to other isoenzymes for practice of the present invention are well known to those skilled in the art.

Fluorescent Dyes. Known fluorescent dyes useful for labeling individual types of capture particles for identification by the flow cytometer include hydrophobic and stable dyes such as those known by the designations IR792 ([2-[2-[3-[(1,3-dihydro-3,3-dimethyl-1-propyl-2H-indol-2-ylidine)ethylidine]-2-(phenylthio)-1-cyclohexen-1-yl]ethenyl]-3,3-dimethyl-1-propylindolium perchlorate]), IR768 ([2-[2-[3-[(1,3-dihydro-3,3-dimethyl-1-propyl-2H-indol-2-ylidine)ethylidine]-2-phenoxy-1-cyclohexen-1-yl]ethenyl]-3,3-dimethyl-1-propylindolium perchlorate])YL22 ([2-[2-[3-[(1,3-dihydro-3,3-dimethyl-1-propyl-2H-benzoindol-2-ylidine)ethylidine]-2-(phenylthio)-1-cyclohexen-1-yl]ethenyl]-3,3-dimethyl-1-propylbenzoindolium iodide]), IR780 ([2-[2-[2-chloro-3-[(1,3-dihydro-3,3-dimethyl-1-propyl-2H-indol-2-ylidine)ethylidine]-1-cyclohexen-1-yl]ethenyl]-3,3-dimethyl-1-propylindolium perchlorate]), JM5488-48 ([2-[2-[2-chloro-3-[(1,3-dihydro-3,3-dimethyl-1-decanyl-2H-benzoindol-2-ylidine)ethylidine]-1-cyclohexen-1-yl]ethenyl]-3,3-dimethyl-1-decanylbenzoindolium iodide]), JM5488-72 ([2-[2-[-3-[(1,3-dihydro-3,3-dimethyl-1-decanyl-2H-benzoindol-2-ylidine)ethylidine]-1-cyclohexen-1-yl]ethenyl]-3,3-dimethyl-1-decanylbenzoindolium iodide]) IR140 and IR143 and their derivatives. Preferred dye structures that can be associated with capture particles as coding indicia include IR792, YL22, and the lipophilic forms of cyanine dyes known under the designations Cy7 (e.g. DiIC₁₈ (7) from Molecular Probes, Inc.) and dibenzoCy7.

Preferred dye structures which can be used to label reporter antibodies for use in the present invention include the cyanine dyes Cy5 and dibenzoCy5. Further examples of useful fluorescent dyes for labeling individual types of particles or for labeling antibodies are known in the art. See, for example, R. P. Haugland, Handbook of Fluorescent Probes and Research Chemicals, 8^{th} Edition, Molecular Probes Inc., Eugene, 1999. For a description of the cyanine dyes Cy5, dibenzoCy5, Cy7, and dibenzoCy7, see Chen, U.S. Pat. No. 5,863,401 and Shen et al., U.S. Pat. No. 6,002,003. Fluorescing cyanine dyes are commercially available from Biological Detection Systems, Inc. (Pittsburgh, Pa.).

Reporter Particles. As described above, labeled reporter antibody can be used to bind to creatine kinase/capture particle complexes. In an alternative embodiment, unlabeled reporter antibodies can be coupled to reporter particles. In this embodiment, the reporter particles contain coding indicia permitting identification of the various types of reporter particles. For example, a reporter particle coupled to CKB reporter antibody (hereinafter, CKB reporter particle) can be labeled with a first fluorescent dye, while a reporter particle coupled to CKM reporter antibody (hereinafter, CKM reporter particle) can be labeled with a second fluorescent dye. In each case, the CKB and CKM reporter particles are labeled so as to be distinguishable from each other and from the CKB and CKM capture particles.

Reporter particles can be formed from the same materials and in the same way as capture particles, but are typically smaller, in the range of 20 nm to 2 μm, preferably from about 40 nm to about 200 nm and more preferably about 60 nm. Preferred dye structures which can be used to label reporter particles for use in the present invention include lipophilic forms of the cyanine dyes Cy5 (e.g. DiIC₁₈ (5) from Molecular Probes, Inc.) and dibenzoCy5.

Reporters. As described above, labeled reporter antibodies can be in the method of the present invention. In another embodiment, unlabeled antibodies coupled to reporter particles are used. As used herein, the term “reporters” refers to labeled antibodies, antibodies coupled to reporter particles, and/or combinations thereof.

Assay and Apparatus. In use, CKB and CKM capture particles are combined with a test biological sample to form an aqueous suspension in which the capture particle antibodies bind to one of the creatine kinase subunits to form capture particle/enzyme complexes. Typically, capture particles are added to a concentration from about 10 particles/ml to about 100,000 particles/ml. Preferably, about 10,000 particles/ml are used, as little cross-linking between the particles occurs in this concentration range.

It is desirable to buffer the aqueous suspension to assure stable test conditions. The type of buffer used can be varied according to the particular analyte or analytes being tested. In a multiplexed assay, it is preferred to use a buffer such as HEPES buffer, which contains no sodium, phosphate or urea, which would interfere with the analysis of electrolytes and/or small metabolite molecules.

CKB and CKM reporters can be added directly to the aqueous suspension. Alternatively, the CKB and CKM capture particle complexes can first be washed to remove unbound creatine kinase enzyme, prior to adding the reporter antibodies or particles. Such a wash step avoids the potential problem of cross-linking several creatine kinase molecules in solution by reporter antibodies. Either way, the reporters interact with the capture particle complexes in a type of “sandwich,” with the creatine kinase enzyme “sandwiched” between the capture particle and the reporter antibody (or reporter particle), each bound to a different subunit of the enzyme.

Care must be taken to avoid binding the reporter antibodies inappropriately to the same creatine kinase subunit as is bound by the capture particle. One way to avoid this difficulty is to use the same antibody for both the capture particle and the reporter antibody, provided this antibody has affinity for a unique epitope on creatine kinase. This also simplifies the assay, as only two antibody types are needed.

The preferred technique for reading the assay according to the invention is flow cytometry, in which the capture particles are read individually as they flow through a reading device. When read, the capture particles are both identified by their coding indicia and measured for interaction with their respective analytes. The measured results thus can be appropriately allocated to the analyte to which they pertain. This permits mixing of capture particles directed to different analytes in a single reagent mixture and simultaneous analysis of multiple analytes in a single test sample.

Because flow cytometry is readily able to distinguish between the capture particles suspended in the test solution and other components of the test solution, the flow cytometer used to carry out the assay procedure of the invention can differentiate between reporter antibodies bound to capture particles and reporter antibodies present in unbound form in the test medium. Consequently, it is not essential to wash the capture particles after incubation with the reporter antibodies and before passage through the flow cytometer.

The reading device includes a first laser, or coding laser, and a first detector for reading the capture particle indicia in order to identify the capture particle as either a CKB capture particle or a CKM capture particle. One laser useful as a coding laser, for example, is a 30 mW diode laser emitting light at a wavelength of about 785 nm.

The reading device can also include a second laser, or assay laser, and second detector for reading the fluorescent dye associated with the reporter antibody bound to the capture particle via bound creatine kinase, to thereby identify the bound reporter antibody as CKB reporter antibody or CKM reporter antibody. A 15 mW diode laser emitting light at a wavelength of about 635 nm, for example, can be useful as the assay laser. Alternatively, a single laser can perform both functions.

These wavelengths lie in the near-infrared wavelength range and are somewhat longer than usual in flow cytometry, but are particularly useful in the present invention because they avoid potential interference in the visible light range caused by substances naturally present in serum or other biological samples. Operation in the near-infrared wavelength range also facilitates the use of laser diodes, which are significantly less expensive than conventional gas lasers.

Additional detectors can be associated with each laser. Such additional detectors would be sensitive to light within a limited range of wavelengths so that signals from different fluorescent dyes can be distinguished.

The detectors can be photomultipliers or photodiodes. Photodiodes are preferred for distinguishing fluorescently coded capture particles both because their solid state nature facilitates miniaturization and operational reliability and because they work better at longer wavelengths in the near-infrared range. Photomultiplier tubes are preferred for analyte quantitation because of their better signal to noise ratio.

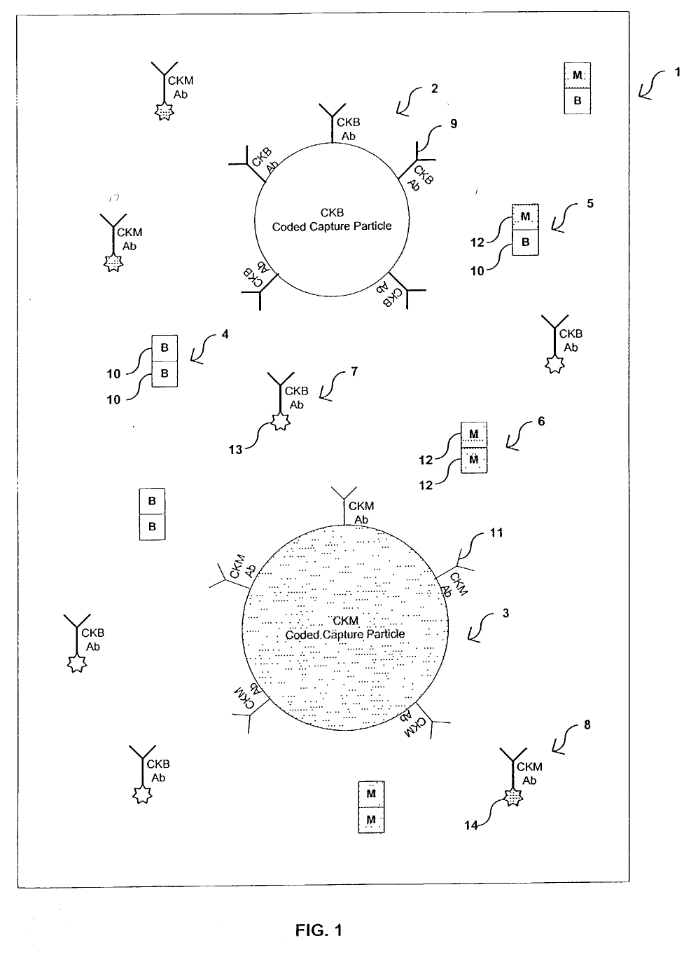

The method of the invention is summarized schematically in FIGS. 1 through 4. In FIG. 1, an assay mix 1 comprises CKB capture particles 2, CKM capture particles 3, CK isoenzymes (in FIG. 1, all three creatine kinase isoenzymes, CK-1 (4), CK-2 (5) and CK-3 (6) are shown, although this may not be the case in an actual biological sample), labeled CKB reporter antibody 7 and labeled CKM reporter antibody 8. It should be appreciated that although FIG. 1 depicts only a single class of analytes, that is, creatine kinase isoenzymes, the method of the present invention can also be used in multiplexed assays. Accordingly, many other analytes, with corresponding uniquely identifiable capture particles, can also be present.

The CKB capture particle 2 is coupled with antibody 9 specific to the B subunit 10 of CK-1 and CK-2. The CKM capture particle 3 is coupled with antibody 11 specific to the M subunit 12 of CK-2 and CK-3. In this example, the two capture particles are coded by size; specifically, the CKM capture particle is larger than the CKB capture particle.

The CKB reporter antibody 7 is labeled with a first fluor, represented by star 13, while the CKM reporter antibody 8 is labeled with a second fluor, represented by star 14.

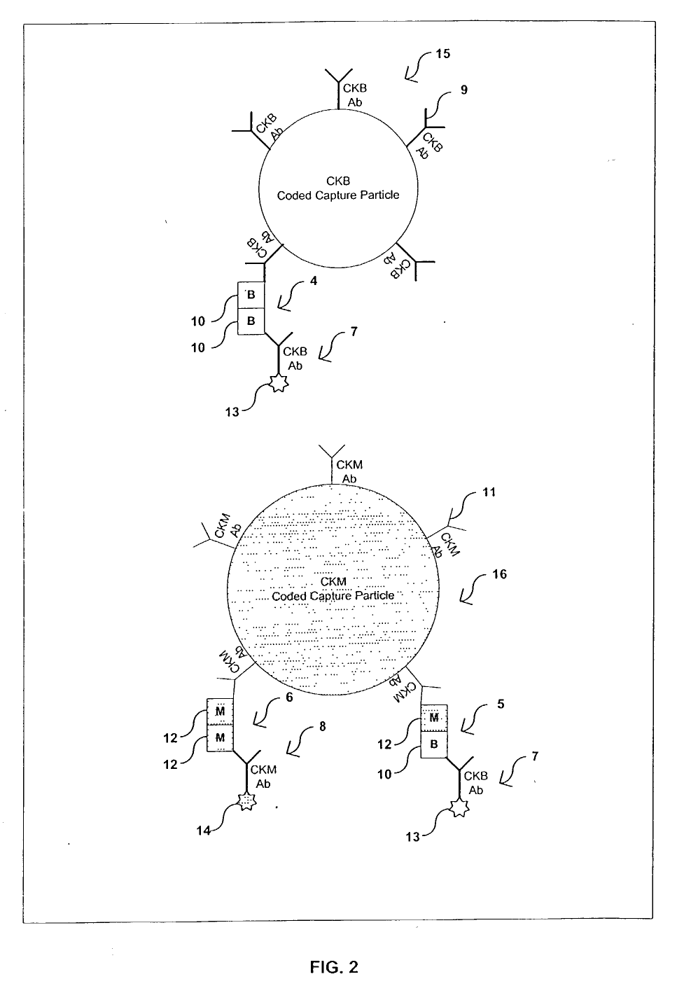

The assay mix is allowed to incubate for a period of time sufficient for each capture particle to interact with the analyte to which it is directed, and, in turn, for the reporter antibodies to interact with the bound analyte. In FIG. 2, two capture particles are shown with bound creatine kinase and bound reporter antibody. The two particles shown are representative only. One skilled in the art will appreciate that other permutations of analyte/reporter antibody binding are possible.

One capture particle complex 15 comprises a CKB capture particle interacting with a single CK-1 enzyme 4, which in turn is interacting with a single CKB reporter antibody 7. As shown, the capture particle CKB antibody 9 interacts with one B subunit of CK-1, while the CKB reporter antibody 7 interacts with the second B subunit. A second capture particle complex 16 comprises a CKM capture particle interacting with the M subunit 12 of a CK-2 enzyme 5, while a CKB reporter antibody 7 interacts with the B subunit 10 of the enzyme. This same capture particle complex 16 is also interacting with CK-3 enzyme 6, which in turn is interacting with CKM reporter antibody 8.

After incubation, the assay mix is transferred to a reading device, where the optical properties, including both the capture particle coding and reporter antibody fluorescence, are read individually for each capture particle. This information is stored and processed to obtain an assay result for the analyte associated with each type of capture particle.

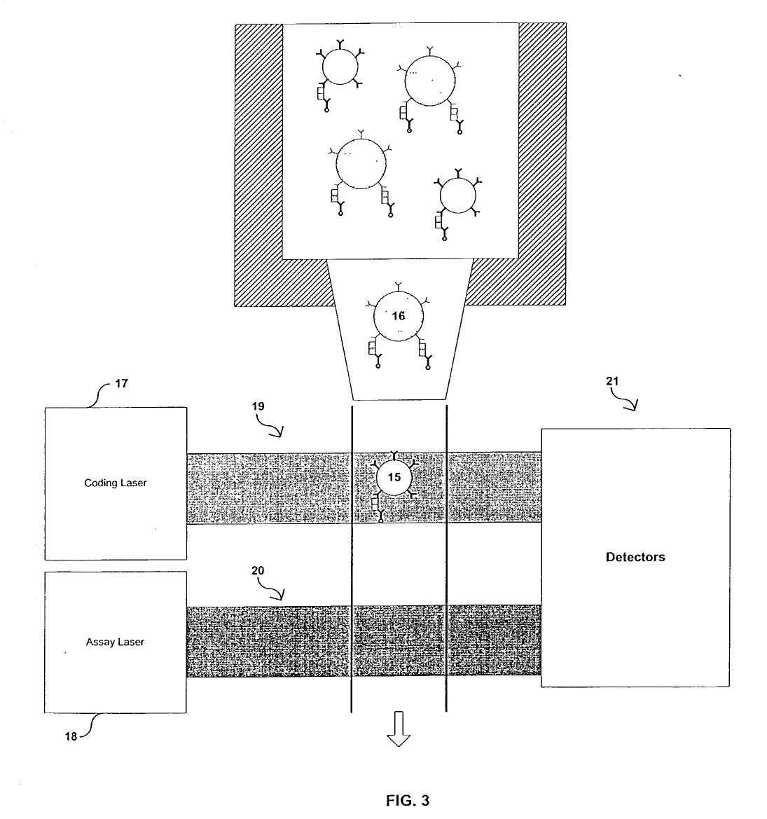

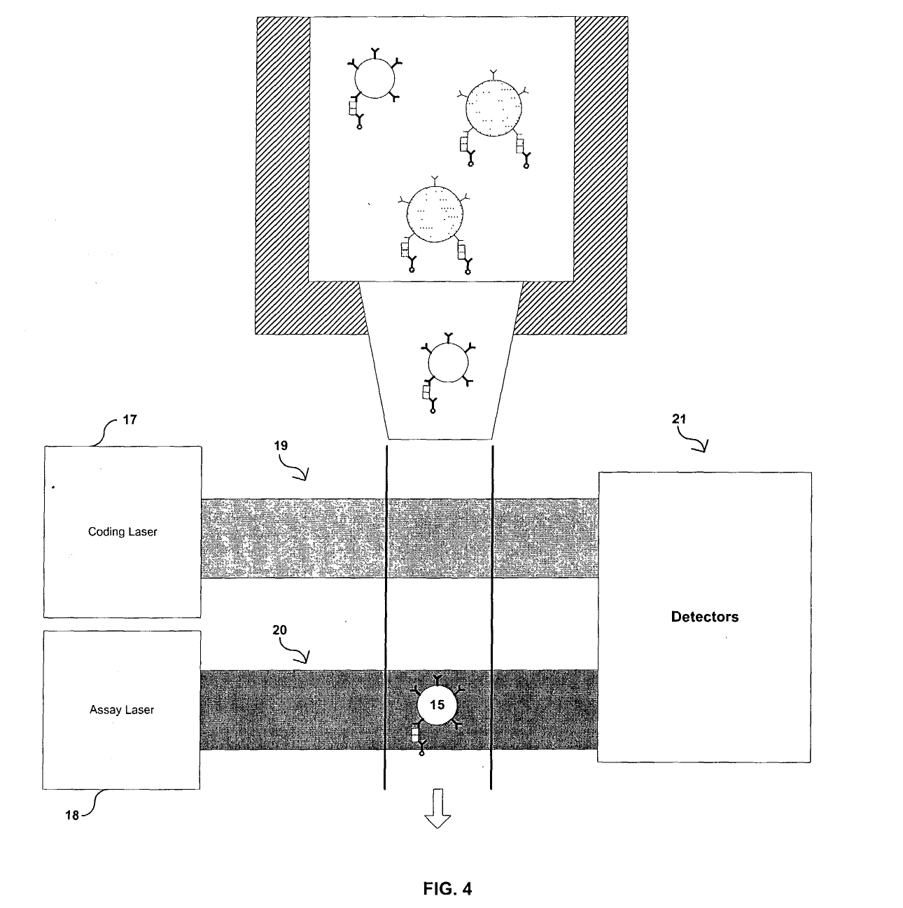

FIGS. 3 and 4 shows an example of such a reading device. In FIG. 3, the capture particle complexes are passed singly through a beam 19 from a coding laser 17 in a flow cytometer. In this example, a single CK capture complex 15 is shown passing through beam 19. The optical signal generated by this passage, in this case, forward and side light scattering, is detected by detectors 21. Information about the light scattering is stored and processed, allowing identification of the capture particle as a CKB capture particle.

In FIG. 4, the CKB complex 15 has continued moving through the flow cytometer and is now shown in the beam 20 from the assay laser 18. During this passage, fluorescence data from the reporter antibody attached to the complex is detected by detectors 21. This fluorescence data is stored and processed, allowing identification of the reporter antibody as, in this case, CKB reporter antibody.

The fluorescence data, when combined with the optical information detected during passage through first beam 19, indicates that capture particle complex 15 contains a CK-1 enzyme.

Reagent Set. The assay method disclosed herein describes several reagents, including capture particles, antibodies, and reporter particles, which are added to the test sample. These reagents can be added to the test sample individually, in various combinations, or all at once. For example, a mixture of two or more kinds of capture particles can be added to the test sample, followed by a mixture of two or more kinds of antibodies (or reporter particles). Alternatively, each kind of capture particle can be added to the test sample separately, followed by the separate addition of each kind of antibody (or reporter particle). In still another embodiment, all of the various capture particles, antibodies and/or reporter particles can be added to the test sample simultaneously. Other combinations are apparent.

As used herein, the term “reagent set” refers to the reagents used in an assay, regardless of whether the reagents are added to the test sample individually or in combination(s). Thus, by way of example, the reagent set for the assay described in FIGS. 1 through 4 comprises CKB capture particles 2, CKM capture particles 3, labeled CKB reporter antibody 7 and labeled CKM reporter antibody 8, irrespective of how these reagents were added to the test sample.

Having generally described the invention, the same will be more readily understood through reference to the following examples, which are provided by way of illustration, and are not intended to be limiting of the present invention.

### Example 1

### Preparing CKB Capture Particles

10 mg of 5.6 μm diameter carboxylate modified polystyrene latex particles (Bang Laboratories) are washed in 1 ml of 50 mM MES buffer, pH 6.1. The wash is repeated once, and the particles are resuspended by sonication in 1 ml of MES buffer. 10 mg of EDAC (1-Ethyl-3-(3-dimethylaminopropyl)-carbodiimide hydrochloride) are added and reacted with the latex particles with continuous mixing for 15 minutes at 20E C to activate the particles.

The activated particles are washed twice in 50 mM MES buffer, adjusted to pH 7.8, and are resuspended by sonication in 0.5 ml of 50 mM MES buffer, pH 7.8. 1.8 mg mouse monoclonal anti-human CKB antibody (Spectral Diagnostics, Inc., Whitestone, Va.) in 0.5 mL of 50 mM MES buffer, pH 7.8, is added and reacted with continuous mixing for 3 hours.

The capture particles are washed and resuspended in 1 ml of 0.03% glycine in 50 mM MES buffer for 30 minutes with gentle mixing. Finally, the capture particles are washed and resuspended in 1% bovine serum albumin in 4 mM Tris buffer at pH 7.4.

### Example 2

### Preparing CKM Capture Particles

CKM capture particles are prepared as described above in Example 1, except that 1.8 mg mouse monoclonal anti-human CKM antibody (Spectral Diagnostics, Inc., Whitestone, Va.) is used instead of anti-human CKB antibody, and 8 mg of 4.5 μm particles (Bangs Laboratories) are used in place of the 5.5 μm particles.

### Example 3

### Preparing CKB Reporter Antibody

1 mg of affinity purified mouse monoclonal anti-human CKB antibody (Spectral Diagnostics, Inc., Whitestone, Va.) is conjugated to Cy5 dye (Fluorolink Cy5, Amersham Pharmacia Biotech, Inc., Piscataway, N.J.) according to the dye manufacturer's directions. The labeled antibody is purified by gel permeation chromatography and suspended in 10 mL Tris buffer at pH 7.4.

### Example 4

### Preparing CKM Reporter Antibody

1 mg of affinity purified mouse monoclonal anti-human CKM antibody (Spectral Diagnostics, Inc., Whitestone, Va.) is conjugated to Cy5.5 dye (Amersham Pharmacia Biotech, Inc., Piscataway, N.J.) according to the dye manufacturer's directions. The labeled antibody is purified by gel permeation chromatography and suspended in 10 mL Tris buffer at pH 7.4.

### Example 5

### Combined Reagent

A dilute aliquot of CKB capture particle and a dilute aliquot of CKM capture particle are each measured in a flow cytometer to establish signature values for forward and side scatter. A combined reagent is produced by mixing equal numbers of CKB and CKM capture particles to a combined concentration of 250,000 beads per ml in 5 mM Tris buffer with 0.01% Tween 20 adjusted to pH 7.4. CKB and CKM reporter antibodies are each added to a final concentration of 12 μg/ml.

### Example 6

### Combined Reagent Reaction

Three sets of six calibrator solutions each are prepared by adding CK-1, CK-2 or CK-3, spanning from one half the minimum to twice the maximum of the reference clinical concentration interval for each isoenzyme. 20 μl aliquots of the combined reagent are mixed in separate reaction vessels with 20 μl of each calibrator solution and with any test samples. 160 μl of Tris buffer at pH 7.4 is added to each reaction vessel. The temperature is maintained within 0.2 degrees of 37 Celsius and the mixtures are stirred gently for 30 minutes before measuring results.

### Example 7

### Measuring the Results of the Reactions

Capture particle signals are measured using a flow cytometer with excitation at 633 nm. Forward scatter and side scatter are detected from the 633 nm source. Fluorescence is detected in two channels with wavelength sensitivity of 650 to 680 nm in the first fluorescence detection channel and of 680 to 720 nm in the second fluorescence detection channel. Data collection is triggered on the forward scatter signal. The detected capture particles are grouped into two clusters of similar forward and side scatter, distinguishing the capture particle types, which are of different sizes.

### Example 8

### Combining the Measured Results

The measured clusters of similar forward and side scatter are associated to CKB or CKM by reference to the scatter measurements made in Example 5. For each reaction, for each CKB- or CKM-associated cluster, a value equal to the median fluorescence from the fluorescence detection channel associated with the signal of that analyte is calculated. This produces a set of four values for each reaction: for the CKB-associated cluster, a median fluorescence value for CK-1 and for CK-2, and for the CKM-associated cluster, a median fluorescence value for CK-2 and for CK-3. The relationship between the values and concentration of each isoenzyme is determined using a curve fit based on the values from the reactions of the calibrator solutions. Each value-isoenzyme concentration pair is fit to a four parameter logistic binding curve to determine curve parameters. The values measured from each of the test samples and the parameters determined from the curve fit are used to calculate the concentration of each isoenzyme from each test sample.

The method of the present invention has been described with reference to measurement of an isoenzyme such as creatine kinase. However, it is contemplated that the reagent mixture of the invention may contain more than the creatine kinase capture particle. For example, the reagent mixture may contain particles designed to quantitatively measure the presence of other analytes such as potassium ions, calcium ions, glucose, urea and the like together with creatine kinase. Thus, the present method provides a method for measuring creatine kinase in a multiplexed assay, in which other analytes are also measured.

All patents and other publications mentioned in this specification are incorporated herein in their entireties by reference.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

What is claimed is:

1. A reagent set for detecting and measuring isoenzymes comprising at least two subunits in a test sample, wherein a first isoenzyme comprises two copies of a first subunit and a second isoenzyme comprises one copy of the first subunit and one copy of a second subunit, the reagent set comprising:
a plurality of sample-insoluble particles having associated therewith immobilized antibody adapted to interact with the first subunit (“first capture particles”);
a plurality of sample-insoluble particles having associated therewith immobilized antibody adapted to interact with the second subunit (“second capture particles”), wherein the first capture particles are optically distinguishable from the second capture particles;
a plurality of reporters adapted to interact with the first subunit (“first reporters”), to thereby produce a first fluorescent signal; and
a plurality of reporters adapted to interact with the second subunit (“second reporters”), to thereby produce a second fluorescent signal.

2. A reagent set according to claim 1, wherein the first reporters comprise antibodies specific to the first subunit (“first antibodies”) and wherein the second reporters comprise antibodies specific to the second subunit (“second antibodies”).

3. A reagent set according to claim 2, wherein the first antibodies and the second antibodies are monoclonal antibodies.

4. A reagent set according to claim 2, wherein the first antibodies are labeled with a first fluorescent dye and the second antibodies are labeled with a second fluorescent dye.

5. A reagent set according to claim 4, wherein the first fluorescent dye is selected from the group consisting of Cy5 and dibenzoCy5.

6. A reagent set according to claim 1, wherein the first reporters comprise sample-insoluble particles having associated therewith immobilized antibody adapted to interact with the first subunit and wherein the reporter particles are labeled with a fluorescent dye.

7. A reagent set according to claim 6, wherein the immobilized antibody is a monoclonal antibody.

8. A reagent set according to claim 6, wherein the fluorescent dye is a lipophilic cyanine dye.

9. A reagent set for measuring creatine kinase (CK) isoenzymes in a test sample, the reagent set comprising:
creatine kinase B (CKB) capture particles comprising a plurality of sample-insoluble particles having associated therewith immobilized antibody adapted to interact with a B subunit of creatine kinase;
creatine kinase M (CKM) capture particles comprising a plurality of sample-insoluble particles having associated therewith immobilized antibody adapted to interact with an M subunit of creatine kinase, wherein the CKM capture particles are optically distinguishable from the CKB capture particles;
a plurality of CKB reporters, adapted to interact with a B subunit of creatine kinase, to thereby produce a first fluorescent signal; and
a plurality of CKM reporters, adapted to interact with M subunit of creatine kinase, to thereby produce a second fluorescent signal.

10. A reagent set according to claim 9, wherein the CKB reporters comprise antibodies specific to the B subunit of creatine kinase and wherein the CKM reporters comprise antibodies specific to the M subunit of creatine kinase.

11. A reagent set according to claim 10, wherein the CKB antibodies and the CKM antibodies are monoclonal antibodies.

12. A reagent set according to claim 10, wherein the CKB antibodies are labeled with a first fluorescent dye and the CKM antibodies are labeled with a second fluorescent dye.

13. A reagent set according to claim 12, wherein the first fluorescent dye is selected from the group consisting of Cy5 and dibenzoCy5.

14. A reagent set according to claim 9, wherein the CKB reporters comprise sample-insoluble particles having associated therewith immobilized antibody adapted to interact with a B subunit of creatine kinase, and wherein the reporter particles are labeled with a first lipophilic fluorescent dye.

15. A reagent set according to claim 14, wherein the immobilized antibody is a monoclonal antibody.

16. A reagent set according to claim 9, wherein the first lipophilic fluorescent dye is a lipophilic cyanine dye.

17. A reagent set according to claim 9, wherein the CKM reporters comprise sample-insoluble particles having associated therewith immobilized antibody adapted to interact with an M subunit of creatine kinase, and wherein the reporter particles are labeled with a second lipophilic fluorescent dye.

18. A reagent set according to claim 17, wherein the immobilized antibody is a monoclonal antibody.

19. A reagent set according to claim 17, wherein the second lipophilic fluorescent dye is a lipophilic cyanine dye.

20. A method for detecting and measuring isoenzymes comprising at least two subunits in a test sample, wherein a first isoenzyme comprises two copies of a first subunit and a second isoenzyme comprises one copy of the first subunit and one copy of a second subunit, the method comprising the steps of:
(a) mixing the test sample with a reagent set comprising:
(1) first capture particles comprising a plurality of sample-insoluble particles having associated therewith immobilized antibody adapted to interact with the first subunit and further comprising coding indicia which confer uniquely identifying optical properties on the first capture particles;
(2) second capture particles comprising a plurality of sample-insoluble particles having associated therewith immobilized antibody adapted to interact with the second subunit and further comprising coding indicia which confer uniquely identifying optical properties on the second capture particles;
(3) a plurality of first reporters, each adapted to interact with the first subunit, to thereby produce a first fluorescent signal; and
(4) a plurality of second reporters, each adapted to interact with the second subunit, to thereby produce a second fluorescent signal;
(b) incubating the resulting mixture for a period of time sufficient for:
(1) the first capture particles to interact with one of the first subunits of the first isoenzyme to form a first capture particle/first isoenzyme capture particle complex and/or with the first subunit of the second isoenzyme to form a first capture particle/second isoenzyme capture particle complex (collectively, “first capture particle complex”);
(2) the second capture particles to interact with the second subunit of the second isoenzyme to form a second capture particle/second isoenzyme capture particle complex (“second capture particle complex”);
(3) the first reporters to interact with the first subunit of the first isoenzyme on the first capture particle/first isoenzyme capture particle complex and/or with the first subunit of the second isoenzyme on second capture particle complex; and
(4) the second reporters to interact with second subunit of the second isoenzyme on second capture particle complex;
(c) reading both the coding optical properties and the fluorescent signal or signals of each capture particle complex individually;
(d) storing the measured fluorescent signal of both the first capture particle complexes and the second capture particle complexes according to the coding optical properties read from the complexes; and
(e) processing the stored measurements for the first capture particle complexes to obtain an assay result for the first isoenzyme and for the second isoenzyme and processing the stored measurements for the second capture particle complexes to obtain an assay result for the second isoenzyme;
whereby a complete chemical analysis of the isoenzyme content of the test sample is obtained.

21. A method according to claim 20, wherein the first reporters comprise antibodies specific to the first subunit (“first antibodies”) and wherein the second reporters comprise antibodies specific to the second subunit (“second antibodies”).

22. A method according to claim 21, wherein the first antibodies and second antibodies are monoclonal.

23. A method according to claim 21, wherein the first antibodies are labeled with a first fluorescent dye and the second antibodies are labeled with a second fluorescent dye.

24. A method according to claim 23, wherein the first fluorescent dye is selected from the group consisting of Cy5 and dibenzoCy5.

25. A method according to claim 20, wherein the first reporters comprise sample-insoluble particles having associated therewith immobilized antibody adapted to interact with the first subunit, and wherein each reporter particle is labeled with a lipophilic fluorescent dye.

26. A method according to claim 25, wherein the immobilized antibody is monoclonal antibody.

27. A method according to claim 25, wherein the lipophilic fluorescent dye is a lipophilic cyanine dye.

28. A method for measuring creatine kinase isoenzymes CK-1, CK-2, CK-3 and total creatine kinase content in a test sample, the method comprising the steps of:
(a) mixing the test sample with a reagent set comprising:
(1) CKB capture particles comprising a plurality of sample-insoluble particles having associated therewith immobilized antibody adapted to interact with a B subunit of creatine kinase and further comprising coding indicia which confer uniquely identifying optical properties on the CKB capture particles;
(2) CKM capture particles comprising a plurality of sample-insoluble particles having associated therewith immobilized antibody adapted to interact with an M subunit of creatine kinase and further comprising coding indicia which confer uniquely identifying optical properties on the CKM capture particles;
(3) a plurality of CKB reporters, each adapted to interact with a CKB subunit, to thereby produce a first fluorescent signal; and
(4) a plurality of CKM reporters, each adapted to interact with a CKM subunit, to thereby produce a second fluorescent signal;
(b) incubating the resulting mixture for a period of time sufficient for:
(1) the CKB capture particles to interact with B subunit of CK-1 to form a CKB/CK-1 capture particle complex and/or with B subunit of CK-2 to form a CKB/CK-2 capture particle complex;
(2) the CKM capture particles to interact with M subunit of CK-2 to form a CKM/CK-2 capture particle complex and/or with M subunit of CK-3 to form a CKM/CK-3 capture particle complex;
(3) the CKB reporters to interact with B subunit of CK-1 on CKB/CK-1 capture particle complexes and/or with B subunit of CK-2 on CKM/CK-2 capture particle complexes; and
(4) the CKM reporters to interact with M subunit of CK-2 on CKB/CK-2 capture particle complexes and/or with M subunit of CK-3 on CKM/CK-3 capture particle complexes;
(c) reading both the coding optical properties and the fluorescent signal or signals of each capture particle complex individually;
(d) storing the measured fluorescent signal of both the CKB capture particle complexes and the CKM capture particle complexes according to the coding optical properties read from the complexes; and
(e) processing the stored measurements for the CKB capture particle complexes to obtain an assay result for CK-1 and for CK-2 and processing the stored measurements for the CKM capture particle complexes to obtain an assay result for CK-2 and for CK-3;
whereby a complete chemical analysis of the creatine kinase content of the test sample is obtained.

29. A method according to claim 28, wherein the CKB reporters comprise antibodies specific to the B subunit of creatine kinase and wherein the CKM reporters comprise antibodies specific to the M subunit of creatine kinase.

30. A method according to claim 29, wherein the CKB antibodies and the CKM antibodies are monoclonal antibodies.

31. A method according to claim 29, wherein the CKB antibody is labeled with a first fluorescent dye and the CKM antibody is labeled with a second fluorescent dye.

32. A method according to claim 31, wherein the first fluorescent dye is selected from the group consisting of Cy5 and dibenzoCy5.

33. A method according to claim 28, wherein the CKB reporters comprise sample-insoluble particles having associated therewith immobilized antibody adapted to interact with a B subunit of creatine kinase, and wherein each reporter particle is labeled with a first lipophilic fluorescent dye.

34. A method according to claim 33, wherein the immobilized antibody is monoclonal antibody.

35. A method according to claim 33, wherein the first lipophilic fluorescent dye is a lipophilic cyanine dye.

36. A method according to claim 28, wherein the CKM reporters comprise sample-insoluble particles having associated therewith immobilized antibody adapted to interact with an M subunit of creatine kinase, and wherein each reporter particle is labeled with a second lipophilic fluorescent dye.

37. A method according to claim 36, wherein the immobilized antibody is monoclonal antibody.

38. A method according to claim 36, wherein the second lipophilic fluorescent dye is a lipophilic cyanine dye.

39. A kit comprising:
an apparatus for assaying multiple isoenzymes in a test sample, and
instructions for use of the apparatus setting forth the method of claim 20.

40. A kit comprising:
an apparatus for assaying creatine kinase isoenzymes and total creatine kinase content in a test sample, and
instructions for use of the apparatus setting forth the method of claim 28.
